(19) United States
(12) Patent Application Publication
(10) Pub. No.: US 2022/0280749 A1
(43) Pub. Date: Sep. 8, 2022

(54) CATHETER HAVING CONNECTOR WITH ELECTRICAL CONDUCTOR CONNECTIONS

(71) Applicant: BIOTRONIK AG, Bülach (CH)

(72) Inventor: Amir Fargahi, Bülach (CH)

(21) Appl. No.: 17/630,219

(22) PCT Filed: Jul. 20, 2020
(86) PCT No.: PCT/EP2020/070428

§ 371 Date: Jan. 26, 2022

(30) Foreign Application Priority Data
Aug. 15, 2019 (EP) 19191879.6

(51) Int. Cl. A61M 25/00

(52) U.S. Cl. CPC A61M 25/0012; A61M 25/0097

(57) ABSTRACT

A catheter includes a catheter shaft with an outer surface extending from a proximal end portion to a distal end portion of the catheter shaft. A connector is connected to the proximal end portion of the catheter shaft. A material surrounds the proximal end portion. A printed circuit board is embedded in the material. The printed circuit board has a conductor track. At least one electrical conductor is printed on the outer surface of the catheter shaft. The at least one electrical conductor contacts the conductor track so that the at least one electrical conductor and the at least one conductor track are electrically conductively connected.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2020/070428, which was filed Jul. 20, 2020, which application claimed priority from European Application Serial Number 19191879.6, which was filed Aug. 15, 2019.

### FIELD OF THE INVENTION

A field of the invention is vascular catheters. A particular application of the invention is to a balloon catheter for performing an angioplasty or implantation of a stent, but the invention is generally applicable and suitable for implementation in any vascular catheter.

### BACKGROUND

Balloon catheters are used, for example, to dilate a stenosis during the course of angioplasty by a stent which is arranged on a balloon of the balloon catheter and is transported by the balloon catheter to the target site where it is dilated by the balloon or is anchored in the vessel to remedy the constriction. The present invention, however, is also applicable to other catheters.

With catheters of this kind there is often the challenge of making suitable electrical contact with an electronic component situated on the catheter or balloon, for example a sensor for measuring the diameter of the balloon.

Often, in complex procedures and soldering processes, electrical lines are laid along the catheter and are connected to electrical connections or plugs at the proximal end of the catheter via solder joints.

Here, the problem with printed electrical conductors in particular is that such electrical conductors made of printed inks are extremely difficult to contact using conventional soldering methods. Due to the high temperatures involved, soldering processes are particularly problematic because of the comparatively temperature-sensitive plastics materials of catheters.

### SUMMARY OF THE INVENTION

A catheter includes a catheter shaft with an outer surface extending from a proximal end portion to a distal end portion of the catheter shaft. A connector is connected to the proximal end portion of the catheter shaft. A material surrounds the proximal end portion. A printed circuit board is embedded in the material. The printed circuit board has a conductor track. At least one electrical conductor is printed on the outer surface of the catheter shaft. The at least one electrical conductor contacts the conductor track so that the at least one electrical conductor and the at least one conductor track are electrically conductively connected.

### BRIEF DESCRIPTION OF THE DRAWINGS

Embodiments of the invention as well as further features and advantages of the invention will be explained hereinafter with reference to the drawings, in which:

FIG. 1 shows a schematic embodiment of a catheter according to the invention,

FIG. 2 shows a perspective view of an outer shaft of the catheter shown in FIG. 1, wherein electrical conductors made of an electrically conductive ink are printed on the outer surface of the outer shaft,

FIG. 3 shows a plan view of a surface of a printed circuit board of the catheter shown in FIGS. 1 and 2, with conductor tracks arranged on the surface,

FIG. 4 shows a schematic view of the arrangement of the printed circuit board on the outer surface of the outer shaft so that the conductor tracks on the surface of the printed circuit board contact and overlap the conductors on the outer surface of the outer shaft (see the detail of FIG. 4),

FIG. 5 shows a schematic view of the finished catheter, wherein a connector, in particular in the form of a Luer lock connector, has been applied to the arrangement according to FIG. 4 by injection moulding, so that the connector surrounds the printed circuit board and the proximal end portion of the outer shaft and fixes the printed circuit board with respect to the outer shaft, while ensuring the electrical contacts between the conductor tracks and their respective associated printed conductors of the outer shaft,

FIG. 6 shows a schematic sectional view of the connector along the line A-A of FIG. 5, and

FIG. 7 shows a further schematic embodiment of a catheter according to the invention for the implantation of a self-expanding stent.

### DETAILED DESCRIPTION OF THE PREFERRED EMBODIMENTS

In accordance with a preferred catheter of the invention it is provided here that the connector includes a material with which the proximal end portion of the catheter shaft is surrounded, at least one electrical conductor being printed on the outer surface of the catheter shaft, the at least one electrical conductor mechanically contacting a conductor track of a printed circuit board which is embedded in the material of the connector, so that the at least one electrical conductor and the at least one conductor track are electrically conductively connected.

According to one embodiment of the invention, the connector is fluidically connected to a lumen of the catheter shaft so that a fluid medium may be introduced into the lumen of the catheter shaft via the connector. This applies in particular if the catheter shaft is formed as the outer shaft of a balloon catheter. In this case, a fluid for filling the balloon is fed into the lumen of the outer shaft via the connector. Alternatively, the connector may be arranged at the proximal end of an inner shaft and may be designed in such a way that a fluid medium may be introduced in order to flush the lumen of the inner shaft.

In the case of a catheter for implanting a self-expanding prosthesis (for example a self-expanding stent), the connector may be arranged at the proximal end of an outer shaft and/or an inner shaft. Also in these cases, the connector may be fluidically connected to the lumen of the outer and/or inner shaft in such a way that a fluid medium may be introduced via the connector in order to flush the lumen.

It is particularly preferably provided that the connector is a Luer lock connector.

The design according to the invention in the aforementioned embodiments thus advantageously supplements the current inflation-deflation function or flushing function of the connectors, in particular the Luer lock connections, with an integrated (connected to the printed circuit board) plug-in connector and, if necessary, a connector belonging to it.

This invention thus provides a highly integrated concept for safe and low-cost electrical connections between conductor tracks and printed electrically conductive inks.

According to a preferred embodiment of the invention, the proximal end portion of the catheter shaft is overmolded with the material of the connector.

Furthermore, according to a preferred embodiment of the invention, the material of the connector is a thermoplastic.

In principle, all thermoplastics suitable for medical devices and which may be processed in an injection moulding process of which the temperature does not affect the functionality of the conductor tracks or a printed circuit board are eligible for the invention. These are in particular ABS (acrylonitrile butadiene styrene), ABS-PC (acrylonitrile butadiene styrene+polycarbonate blend), ANMA (acrylonitrile methyl acrylate copolymer), CAP (cellulose acetate propionate), COC (cyclic olefin copolymer), copolyesters, LCP (liquid crystal polyester), MABS (methylmethacrylate acrylonitrile butadiene styrene), polyamides (transparent, PA11, PA12, PA6, PA66), PBT (polybutylene terephthalate), PC (polycarbonate), PC+polyester (polycarbonate+polyester blend), PCTA (polycyclohexylenedimethylene terephthalate/isophthalate copolyester), PCTG (polycyclohexylenedimethylene terephthalate glycol copolymer), HDPE (high-density polyethylene), LLDPE (linear low-density polyethylene), MDPE (medium-density polyethylene), PEBA (polyester block amide, thermoplastic polyamide-polyether elastomer with different Shore hardness), PEI (polyether imide), PEI+PCE (polyetherimide+polycarbonate ester blend), PET (polyethylene terephthalate), PFA (perfluoroalkoxyethylene), PGA (polyglycolic acid), PLA (polylactic acid), PMMA (poly(methyl methacrylate)), PMP (polymethylpentene), POM (polyoxymethylene), PP (polypropylene), PPE+PS (polyphenylene ether+polystyrene), PPSU (polyphenylsulfone), PS (polystyrene), PSU (polysulfone), PVC (polyvinyl chloride, plasticised), PCC-P (polyvinyl chloride elastomer, plasticised), PVDF (polyvinylidene difluoride), SAN (acrylonitrile styrene copolymer), SB (styrene butadiene copolymer), SEBS (styrene ethylene butylene styrene block copolymer, various hardness), SMMA (styrene methyl methacrylate copolymer), SRP (self-reinforcing polyphenylene), TPC (thermoplastic copolyester-ether elastomer of various hardness)), TPU (thermoplastic polyurethane elastomer of various hardness), TPV (thermoplastic vulcanisate of various hardness).

Furthermore, according to a preferred embodiment of the invention, it is provided that the at least one electrical conductor and the conductor track are arranged overlapping, wherein preferably the conductor track and the at least one electrical conductor on the outer surface of the catheter shaft overlap each other in a common direction of longitudinal extent, i.e. in particular are not arranged crossing each other.

Furthermore, in accordance with a preferred embodiment of the invention, it is provided that the conductor track is arranged on a surface of the printed circuit board in a region of the conductor track in which the conductor track overlaps with the at least one electrical conductor, the surface facing or being arranged on the outer surface of the catheter shaft.

Away from the overlap or contact between the at least one electrical conductor and the conductor track, the at least one electrical conductor and the at least one conductor track are preferably suitably electrically insulated. In the case of the printed circuit board, this insulation may be ensured by the surrounding material of the connector.

Furthermore, according to a preferred embodiment of the invention it is provided that the printed circuit board has a first and a second portion connected thereto, the two portions being arranged at an angle to each other, in particular at right angles. This configuration allows a particularly simple contacting or connection of the catheter, in particular via a plug-in connector, with a device for the application, evaluation and/or control of electrical signals.

Furthermore, according to a preferred embodiment of the invention, said region of the conductor track is situated at an end of the first portion.

Furthermore, according to a preferred embodiment of the invention, it is provided that the printed circuit board is connected to a plug-in connector, via which the conductor track may be contacted electrically conductively. Preferably, the plug-in connector is connected to an end portion of the second portion of the printed circuit board, in particular by a solder connection. Due to the angular arrangement or embodiment of the two portions of the printed circuit board, the plug-in connector may be led out of the connector particularly transversely or at an angle to the axial direction of the catheter.

Furthermore, according to a preferred embodiment of the invention it is provided that the conductor track in said region of the conductor track overlapping the at least one electrical conductor has a width greater than a width of the at least one electrical conductor. In each case, the width perpendicular to the direction of longitudinal extent of the conductor track or the conductor is meant here.

Furthermore, according to a preferred embodiment of the invention it is provided that the printed circuit board is formed from polyimide. Furthermore, the printed circuit board may have a thickness in the range of, for example, from 0.03 mm to 0.1 mm.

Preferably, the catheter is a balloon catheter, wherein in particular the at least one electrical conductor is connected to a sensor of the balloon catheter, in particular to a sensor arranged on a balloon of the balloon catheter.

Above, the invention has been described on the basis of one conductor track and one printed electrical conductor of the catheter shaft. Of course, a plurality of separate conductor tracks of the printed circuit board and a plurality of printed conductors of the catheter shaft may be in mechanical and thus electrically conductive contact with each other in the manner described herein.

Another aspect of the present invention relates to a method for producing a catheter (in particular for producing a catheter according to the invention), including the steps of:
printing at least one electrical conductor on an outer surface of a catheter shaft of the catheter using an electrically conductive ink,
providing a printed circuit board which includes at least one conductor track,
arranging the printed circuit board on the outer surface of the catheter shaft in the region of a proximal end portion of the outer shaft so that the conductor track mechanically and thus electrically conductively contacts the at least one electrical conductor and in particular overlaps with it, and
surrounding the printed circuit board and the proximal end portion of the catheter shaft with a material for forming a connector connected to the proximal end portion of the outer shaft.

With regard to the arrangement of the printed circuit board in relation to the outer surface of the catheter shaft, it may also be provided that the printed circuit board is fixed on the outer surface, for example by shrink-on tubing.

According to an embodiment of the method, it is preferably provided that a plug-in connector is connected to the printed circuit board so that the conductor track (and thus later the at least one printed electrical conductor) may be electrically contacted via the plug-in connector, wherein in particular the plug-in connector is connected to the printed circuit board before the printed circuit board is arranged on the outer surface of the catheter shaft (and in particular before the printed circuit board and the proximal end portion of the catheter shaft are surrounded by the material of the connector).

In accordance with a further embodiment of the method, it is provided that, in order to surround the proximal end portion of the catheter shaft and the printed circuit board with the material of the connector, the proximal end portion of the catheter shaft and the printed circuit board are arranged in a mould (for example an injection mould) and are surrounded in the mould with the material, in particular are overmoulded by injection moulding, i.e. they are cast into the material by injection moulding.

Of course, this method also allows a plurality of conductor tracks of the printed circuit board to be connected or contacted with one electrical conductor of the catheter shaft each.

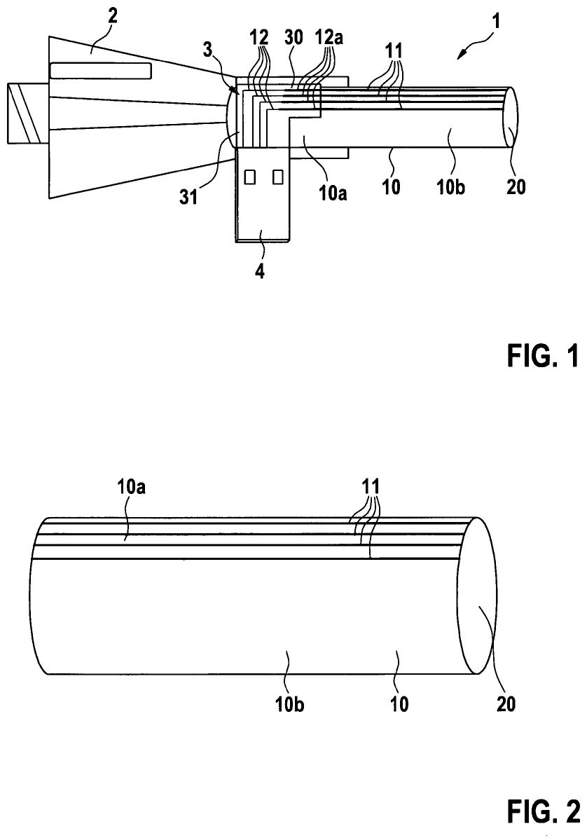

FIG. 1 shows, in conjunction with FIG. 2, an embodiment of a catheter 1 according to the invention, with an outer shaft 10 extending from a proximal end portion 10a to a distal end portion (cut off, not shown) of the outer shaft 10 and having an outer surface 10b. The terms ‘proximal’ and ‘distal’ in this case mean, in the usual manner, that the proximal end portion 10a is closer to the physician or user of catheter 1, the outer shaft 10 being inserted into the patient's vessel with the distal end portion first.

The catheter 1 further includes a connector 2, which is connected to the proximal end portion 10a of the outer shaft 10 or is injected-moulded or suitably moulded integrally onto it and is designed to establish a fluid-conducting flow connection to a corresponding further connector.

The connector 2 may, for example, be designed as a Luer lock connector and is fluidically connected to a lumen 20 of the outer shaft 10, through which, for example, a fluid may be introduced into a balloon connected distally to the outer shaft 10 (for example in order to unfold the balloon).

Preferably, the connector 2 includes or is formed from a material processed for example by injection moulding or a comparable moulding process, in which the proximal end portion 10a of the outer shaft 10 is embedded together with a printed circuit board 3, wherein electrical conductors 11 are printed on the outer surface 10b of the outer shaft 10 and each contact an associated conductor track 12 of the printed circuit board 3, so that the conductors 11 are electrically conductively connected to their respective associated conductor tracks 12. The conductors 11 are conductors 11 printed from an electrically conductive ink.

As may be seen in particular from the detail in FIG. 4, it is provided that the electrical conductors 11 and the conductor tracks 12 are arranged overlapping, more specifically preferably in the common direction of longitudinal extent.

In other words, the conductor tracks 12 each have a region 12a, in which the conductor track 12 is arranged on a surface 3a of the printed circuit board 3 and overlaps there with the associated printed conductor 11, so that electrically conductive contacts result between the conductor tracks 12 and the conductors 11. Here, the surface 3a of the printed circuit board 3 faces the outer surface 10b of the outer shaft 10 on which the conductors 11 are printed. In FIGS. 1 and 4, the printed circuit board 3 would actually cover the underlying portions of the conductors 11 and conductor tracks 12. These are nevertheless drawn in to make the structure visible.

The printed circuit board 3 preferably has an angular shape, with a first portion 30 of printed circuit board 3 running at an angle, in particular at right angles, to a second portion 31 of the printed circuit board 3.

Said overlapping regions 12a are provided here at one end of the first portion 30, whereas an end portion of the second portion 31 of the printed circuit board 3 is connected to a plug-in connector 4, via which the conductor tracks 12 (and thus the conductors 11) may be electrically conductively contacted. Preferably, the conductor tracks 12, at least in the regions 12a, have a width which is greater than a width of the conductors 11 perpendicular to the direction of longitudinal extent of the conductors 11 or conductor tracks 12.

For example, the printed circuit board 3 may be formed from a polyimide and may have a thickness in the range of from 0.03 mm to 0.1 mm, in particular 0.05 mm. The conductor tracks 12 may be etched in a known manner.

Preferably, the male or female connector 4 is prepared in advance with a printed circuit board 3. Through the injection moulding process (or a comparable moulding process to produce the connector 2), the contact connections between the conductor tracks 12 of the printed circuit board 3 and the printed ink lines 11 are realised or maintained due to the pressure and the form fit of the material of the connector 2.

A possible sequence of the method for producing the catheter 1 is shown in FIGS. 2 to 6.

First, the outer shaft 10 of the catheter 1 may be prepared by printing the necessary conductors 11 onto the outer surface 10b of the outer shaft using electrically conductive ink. The width, thickness and ink material may be varied according to requirements (see FIG. 2).

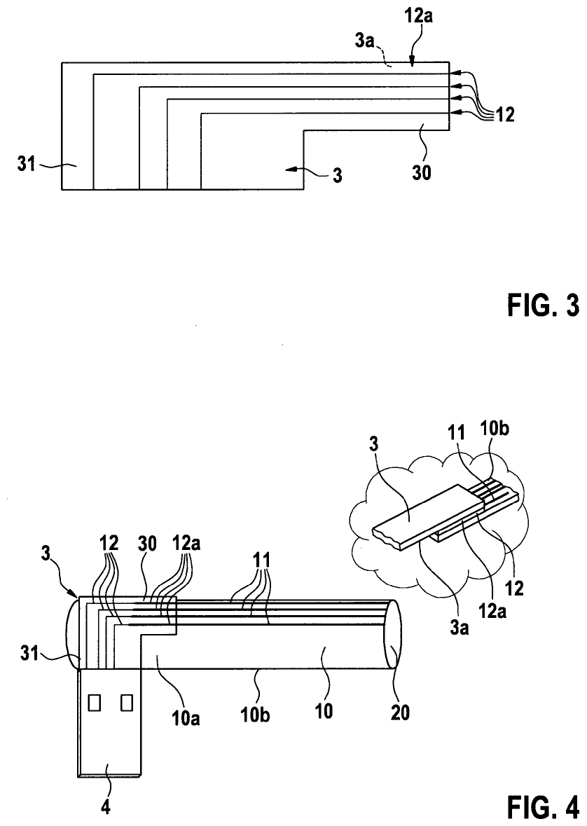

Then, the printed circuit board 3 is provided (for example made of polyimide foil, 0.05 mm thick) and the necessary conductor tracks 12 are etched or formed on it. The width of the conductor tracks 12 should preferably be greater than the width of the printed lines 11 (see FIG. 3).

Furthermore, the printed circuit board 3 is connected to a plug-in connector 4 (for example by soldering). For example, an HDMI-D plug with a width of 6.4 mm and a height of 2.8 mm may be used as plug-in connector 4.

The printed circuit board 3 is then fixed together with the plug-in connector 4 as shown in FIG. 4 on the outer surface 10b of the outer shaft 10 (for example using shrink-on tubing), so that the above-described overlap between the conductor tracks 12 and the printed lines 11 on the outer shaft 10 results.

The prepared arrangement according to FIG. 4 (printed circuit board 3 with plug-in connector 4 and outer shaft 10) is lastly placed in an injection mould and the connector 2 is injection-moulded onto the assembly. The shrink-on tubing, preferably made of thin polytetrafluoroethylene, is not removed but is overmoulded as well.

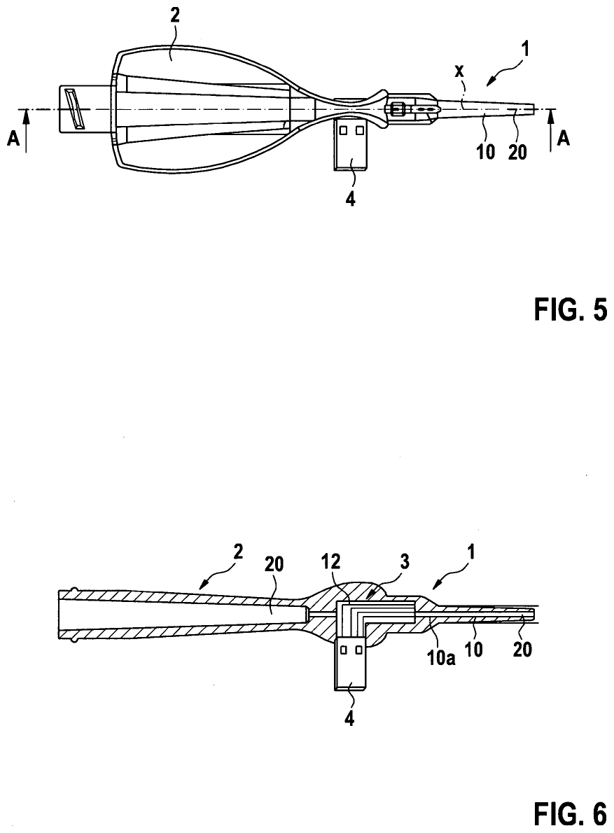

A possible design of connector 2 is shown in FIGS. 5 and 6, wherein the connector 2 is embodied as a Luer lock connector. Other connector types may of course also be realised with this invention.

FIGS. 5 and 6 each show the plug-in connector 4 schematically extended in the drawing plane for better visibility. In FIG. 6, the plug-in connector 4 would actually extend perpendicularly to the drawing plane.

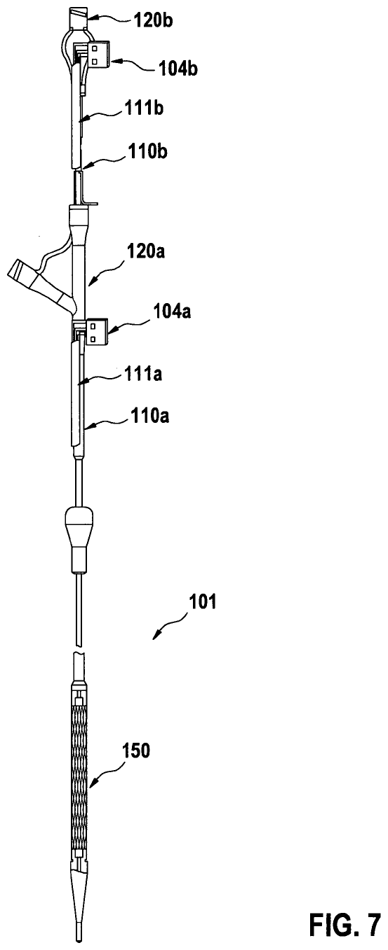

FIG. 7 shows an embodiment of the invention as a catheter 101 for the implantation of a self-expandable stent 150. The catheter 101 has two catheter shafts 110a and 110b which are movable relative to each other, wherein the self-expanding stent 150 situated between the outer and inner shaft 110b is released by retracting the outer shaft 110a.

In this embodiment of the invention, both the outer shaft 110a and the inner shaft 110b have a connector 120a and 120b respectively at their proximal end. The two connectors 120a, 120b are configured such that a fluid for flushing the lumen may be introduced via them (connector 120a is designed to supply a fluid for flushing the lumen between the outer shaft 110a and the inner shaft 110b, connector 120b is designed to supply a fluid for flushing the lumen of the inner shaft 110b).

The connectors 120a and 120b are designed analogously to the embodiment according to FIGS. 1 to 4 for connecting the printed electrical conductors 111a and 111b on the catheter shafts 110a and 110b via a printed circuit board to their respective plug-in connectors 104a and 104b.

The present invention offers the advantage of a “two in one design” in which, for example, a Luer connection having a second function (electrical connection between printed circuit board/plug-in connector and printed lines) is provided, which improves then handling for the physician.

The invention further enables a secure electrical connection between catheter-side printed lines and an external component to be connected thereto (for example measuring device, power supply, etc.).

1. A catheter comprising:
a catheter shaft with an outer surface extending from a proximal end portion to a distal end portion of the catheter shaft, and
a connector connected to the proximal end portion of the catheter shaft,
a material surrounding the proximal end portion,
a printed circuit board embedded in the material, the printed circuit board having a conductor track,
at least one electrical conductor printed on the outer surface, the at least one electrical conductor contacting the conductor track so that the at least one electrical conductor and the at least one conductor track are electrically conductively connected.

2. The catheter according to claim 1, wherein the at least one electrical conductor comprises a cured electrically conductive ink.

3. The catheter according to claim 1, wherein the proximal end portion is overmoulded with the material.

4. The catheter according to claim 1, wherein the material is a thermoplastic material.

5. The catheter according to claim 1, wherein the at least one electrical conductor and the conductor track overlap.

6. The catheter according to claim 1, wherein the conductor track is arranged on a surface of the printed circuit board in a region of the conductor track that overlaps with the at least one electrical conductor, and the surface of the printed circuit board faces the outer surface.

7. The catheter according to claim 1, wherein the printed circuit board has a first portion and a second portion arranged at an angle to each other.

8. The catheter according to claim 7, wherein the region of the conductor track is situated at an end of the first portion of the printed circuit board.

9. The catheter according to claim 1, wherein the printed circuit board is connected to a plug-in connector via which the conductor track can be contacted electrically conductively.

10. The catheter according to claim 9, wherein the plug-in connector is connected to an end portion of the printed circuit board.

11. The catheter according to claim 6, the conductor track in said region has a width greater than a width of the at least one electrical conductor.

12. The catheter according to claim 1, wherein the printed circuit board is formed from polyimide.

13. The catheter according to claim 1, wherein the catheter shaft is an outer shaft of a balloon catheter and the connector is configured to establish a fluid-conducting flow connection to a another connector.

14. A method for producing a catheter, comprising the steps of:
printing at least one electrical conductor on an outer surface of a catheter shaft of the catheter using an electrically conductive ink,
arranging printed circuit board comprising at least one conductor track on the outer surface of the catheter shaft in the region of a proximal end portion of the catheter shaft so that the conductor track contacts the at least one electrical conductor, and
surrounding the printed circuit board and the proximal end portion of the catheter shaft with a material to form a connector connected to the proximal end portion of the catheter shaft.

15. The method according to claim 14, wherein a plug-in connector is connected to the printed circuit board so that the conductor track can be electrically contacted via the plug-in connector, wherein the plug-in connector is connected to the printed circuit board before the printed circuit board is arranged on the outer surface of the proximal end portion of the catheter shaft.

16. The method according to claim 14, wherein the surrounding comprising arranging the proximal end portion of the catheter shaft and the printed circuit board in an injection mould and overmoulding with the material in the injection mould.

17. The catheter according to claim 1, wherein the printed circuit board has a thickness in the range of from 0.03 mm to 0.1 mm.
